(19) United States
(12) Patent Application Publication
(10) Pub. No.: US 2010/0087922 A1
(43) Pub. Date: Apr. 8, 2010

(54) Kit and Method for Comparison of Breast Implants

(75) Inventor: Robert S. Hamas, Dallas, TX (US)

Correspondence Address: THE WEBB LAW FIRM, P.C., 700 KOPPERS BUILDING, 436 SEVENTH AVENUE, PITTSBURGH, PA, 15219, US

(73) Assignee: IDEAL IMPLANT INCORPORATED, Dallas, TX (US)

(21) Appl. No.: 12/246,779

(22) Filed: Oct. 7, 2008

(51) Int. Cl. A61F 2/12

(52) U.S. Cl. 623/8

(57) ABSTRACT

A kit for comparing the physical properties of different breast implants comprising a plurality of display structures supporting a curved cylindrical surface.

### BACKGROUND OF THE INVENTION

1. Field of the Invention

This invention relates to the field of plastic surgery and, more particularly, to the comparison of the physical properties of different breast implants.

2. Description of Related Art

Breast implants typically are comprised of a flexible shell filled with a liquid. Various models or types with different physical properties are available commercially and others are described in the patent and trade literature. Some are silicone gel filled and others are saline solution filled. They may be fully filled with, say, 200, 400, or 600 cubic centimeters of liquid. For various reasons, they may be less or more than fully filled. They may have various shapes, such as round or teardrop when viewed from the front or side, and may have various profiles, such as high, moderate or low. The implant shell may vary in thickness or texture. A surgeon's ability to compare different physical properties of breast implants by placing them within a patient at the time of surgery is limited since they are partially hidden and/or distorted by the tissues and/or surgical instruments. Typically, comparison is made by simply resting two different implants side by side on a flat surface. While this is the method commonly used by surgeons and by breast implant manufacturers, it does not accurately provide information on how the different implants will contour to the chest wall since the chest wall is generally cylindrical with a generally vertical cylindrical axis at the location where they are implanted. Furthermore, the radius of curvature of the chest wall may be different for different patients and also may be different at different points on the chest wall of the same patient, making it desirable to compare various models and types of breast implants on cylindrical surfaces to facilitate selection of the optimal implant for any given patient.

### SUMMARY OF THE INVENTION

Briefly, according to one embodiment of this invention, there is provided a kit for comparing various models or types of breast implants with different physical properties comprising a plurality of identical structures, the structures supporting a curved cylindrical surface having a radius or radii of curvature between 7 and 18 centimeters, the length of the directrix of the curved cylindrical surface being such that structures can be rested on a surface with the curved cylindrical surface facing outward. The curved cylindrical surface may have a directrix that is a circle, an ellipse, a parabola, a hyperbola, or a combination of tangent portions of each. The structures may comprise shells or may comprise solids with opposed curved cylindrical and flat surfaces. The structures may comprise a segment of a circular cylinder having a flat face opposed to the circular cylindrical face.

Briefly, according to another embodiment of this invention, there is provided a kit for comparing various models or types of breast implants with different physical properties comprising a plurality of structures, each structure supporting a curved cylindrical surface having a different radius or radii of curvature between 7 and 18 centimeters, the length of the directrix of the curved cylindrical surface being such that structures can be rested on a surface with the curved cylindrical surface facing outward. The curved cylindrical surface may have a directrix that is a circle, an ellipse, a parabola, a hyperbola, or a combination of tangent portions of each. The structures may comprise shells or may comprise solids with opposed curved cylindrical and flat surfaces. The structures may comprise a segment of a circular cylinder having a flat face opposed to the circular cylindrical face.

Briefly, according to another embodiment of this invention, there is provided a kit for comparing two or more of a particular model or type of breast implant when each one contains a different fluid volume comprising a plurality of identical structures, each structure supporting a curved cylindrical surface having a radius or radii of curvature between 7 and 18 centimeters, the length of the directrix of the curved cylindrical surface being such that structures can be rested on a surface with the curved cylindrical surface facing outward. The curved cylindrical surface may have a directrix that is a circle, an ellipse, a parabola, a hyperbola, or a combination of tangent portions of each. The structures may comprise shells or may comprise solids with opposed curved cylindrical and flat surfaces. The structures may comprise a segment of a circular cylinder having a flat face opposed to the circular cylindrical face.

Briefly, according to another embodiment of this invention, there is provided a kit for comparing two or more of a particular model or type of breast implant when each one contains a different fluid volume comprising a plurality of structures, each structure supporting a curved cylindrical surface having a different radius or radii of curvature between 7 and 18 centimeters, the length of the directrix of the curved cylindrical surface being such that structures can be rested on a surface with the curved cylindrical surface facing outward. The curved cylindrical surface may have a directrix that is a circle, an ellipse, a parabola, a hyperbola, or a combination of tangent portions of each. The structures may comprise shells or may comprise solids with opposed curved cylindrical and flat surfaces. The structures may comprise a segment of a circular cylinder having a flat face opposed to the circular cylindrical face.

Briefly, according to another embodiment of this invention, there is provided a method of comparing various models or types of breast implants with different physical properties comprising the steps of placing each on identical structures with a curved cylindrical surface having a radius or radii of curvature between 7 and 18 centimeters and resting the structures on a flat surface and/or rotating the structures around any axis and/or tilting the structures to any degree and/or holding each implant against the surfaces to simulate the overlying soft tissues of the body and keep the implants in place on the surface.

According to another method embodiment, there is provided a method of comparing various models or types of breast implants with different physical properties comprising the steps of placing each on a structure with a curved cylindrical surface having a different radius or radii of curvature between 7 and 18 centimeters and resting the structures on a flat surface and/or rotating the structures around any axis and/or tilting the structures to any degree and/or holding each implant against the surfaces to simulate the overlying soft tissues of the body and keep the implants in place on the surface.

According to another method embodiment, there is provided a method of comparing two or more of a particular model or type of breast implant when each one contains a different fluid volume comprising the steps of placing each on identical structures with a curved cylindrical surface having a radius or radii of curvature between 7 and 18 centimeters and resting the structures on a flat surface and/or rotating the structures around any axis and/or tilting the structures to any degree and/or holding each implant against the surfaces to simulate the overlying soft tissues of the body and keep the implants in place on the surface.

According to another method embodiment, there is provided a method of comparing two or more of a particular model or type of breast implant when each one contains a different fluid volume comprising the steps of placing each on a structure with a curved cylindrical surface having a different radius or radii of curvature between 7 and 18 centimeters and resting the structures on a flat surface and/or rotating the structures around any axis and/or tilting the structures to any degree and/or holding each implant against the surfaces to simulate the overlying soft tissues of the body and keep the implants in place on the surface.

### BRIEF DESCRIPTION OF THE DRAWINGS

Further features and other objects and advantages may become clear from the following description of preferred embodiments made with reference to the drawings in which:

FIG. 1 shows the cross sections of two identical shell structures having circular cylindrical surfaces for comparing breast implants according to this invention;

FIG. 2 shows the cross sections of two identical solid structures having elliptical cylindrical surfaces for comparing breast implants according to this invention;

FIG. 3 shows the cross sections of two shell structures with circular cylindrical surfaces having different radii of curvature for comparing breast implants according to this invention;

FIG. 4 illustrates an embodiment of a kit comprising pairs of display structures; and

FIG. 5 illustrates an embodiment of a kit comprising a plurality of display structures having different radii of curvature.

### DESCRIPTION OF THE PREFERRED EMBODIMENTS

For purposes of this application, a cylindrical surface is generated by a line (the genetrix) which moves so that it always intersects a plane curve (the directrix) and always remains parallel to a fixed line that lies outside the plane of the curve. Any of the positions assumed by the moving line (the genetrix) as it generates the surface is called an element of the latter. A circular cylindrical surface has a genetrix that is a circle.

Referring to FIG. 1, there is shown a kit comprising two identical display structures for comparing breast implants with different physical properties. The structures 10 and 11 shown in FIG. 1 are portions of circular cylindrical shells placed side by side on a flat surface 12. The radius of curvature of the shells is indicated by the arrow R. Resting on each shell is a representation of a breast implant. Note that the edge of the implant illustrated on the left lies higher above the surface than the edge of the implant on the right. This difference in physical properties may be of particular importance to a surgeon in determining which implant is most appropriate for a particular patient. The manner in which the implant edges lie against the cylindrical surface of the structure gives an indication of the likely manner in which the implant edges will lie against the chest wall after implantation surgery and also how palpable they will be.

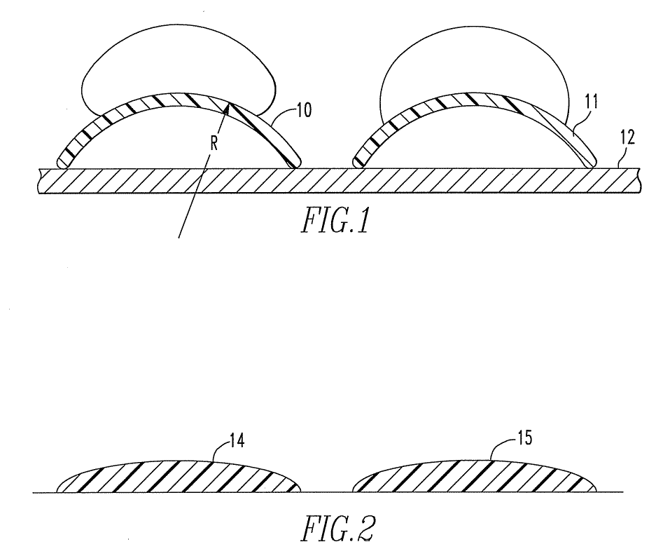

Referring to FIG. 2, there is shown a kit comprising two identical solid structures having a cylindroid surface and a flat surface. The cylindroid surface has an ellipse for a directrix.

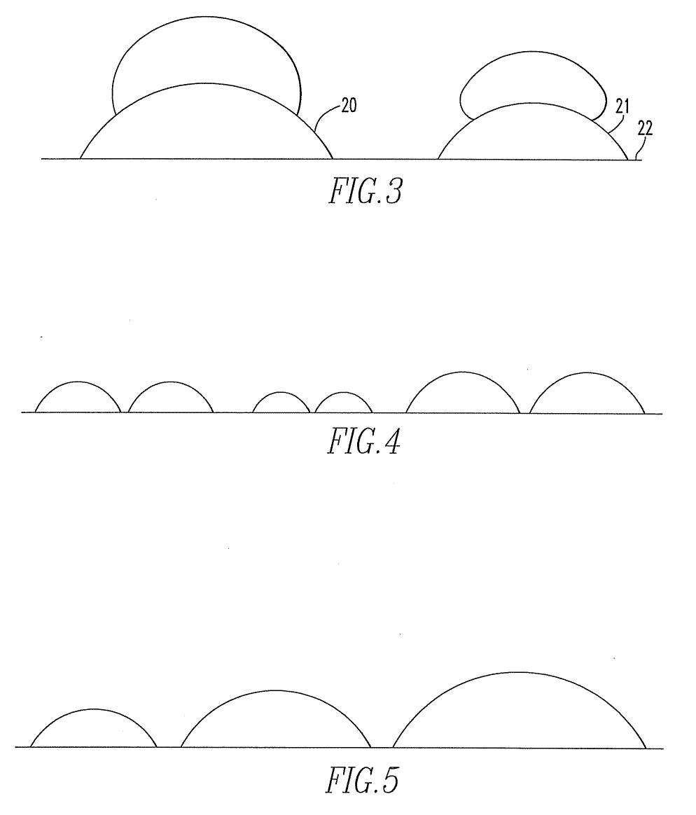

Referring to FIG. 3, there is shown a kit comprising two display structures having different radii of curvature for comparing breast implants with different physical properties. The structures 20 and 21 shown in FIG. 3 are portions of circular cylindrical shells placed side by side on a flat surface 22. Resting on each shell is a representation of a breast implant.

Referring to FIG. 4, the kit may comprise a plurality of display structures each having a different maximum radius of curvature.

Referring to FIG. 5, the kit may comprise a plurality of matched pairs of display structures each having a different maximum radius of curvature.

According to a preferred embodiment of this invention, the kit may comprise a plurality of display structures each having identical curved cylindrical surfaces.

Having thus defined my invention with the detail and particularity required by the Patent Laws, what is desired protected by Letters Patent is set forth in the following claims.

The invention claimed is:

1. A kit for comparing the physical properties of different breast implants comprising a plurality of display structures, said structures supporting a curved cylindrical surface having a radius or radii of curvature between 7 and 18 centimeters, the length of the directrix being such that each structure can be rested on a surface with the curved cylindrical surface facing outward without rocking.

2. The kit according to claim 1, wherein the display structures comprise segments of curved cylinders having a flat face opposed to the curved cylindrical face.

3. The kit according to claim 1, wherein the display structures comprise shell segments.

4. The kit according to claims 1, 2, or 3, wherein the display structures have curved cylindrical surfaces having a directrix that is a cycle, an ellipse, a parabola, a hyperbola, or a combination of tangent portions of one or more of each.

5. The kit according to claims 1, 2, 3, or 4, comprising at least two display structures having identical shapes.

6. The kit according to claims 1, 2, 3, or 4, comprising at least two display structures having different radii of curvature.

7. The kit according to claim 6, comprising at least three display structures each having different maximum radius of curvature.

8. A method of comparing at least two breast implants comprising the step of placing each on an identical curved cylindrical surface having a radius or radii of curvature between 7 and 18 centimeters and observing the shape assumed by the implants.

9. A method of comparing at least two breast implants comprising the step of placing each on curved cylindrical surfaces having different maximum radius of curvature.

10. The method according to claim 8 or 9, further comprising a step of holding the implants against the curved cylindrical surfaces while placing the surfaces in a tilted position.

11. The method according to claim 8 or 9 for comparing at least two breast implants of a particular model or type containing different volumes of fluid.
